(19) United States
(12) Patent Application Publication
(10) Pub. No.: US 2019/0015084 A1
(43) Pub. Date: Jan. 17, 2019

(54) TARGETING CUBES FOR MRI BIOPSY DEVICE

(71) Applicant: Devicor Medical Products, Inc., Cincinnati, OH (US)

(72) Inventors: Bryan R. Keller, Loveland, OH (US); Melody L. Stamper (formerly Mitro), Batavia, OH (US)

(21) Appl. No.: 15/755,658

(22) PCT Filed: Aug. 30, 2016
(86) PCT No.: PCT/US16/49361

§ 371 Date: Feb. 27, 2018

Related U.S. Application Data
(60) Provisional application No. 62/212,191, filed on Aug. 31, 2015.

(51) Int. Cl. A61B 10/02; A61B 5/055; A61B 90/00

(52) U.S. Cl. CPC A61B 10/0283; A61B 2010/009; A61B 90/03; A61B 5/0555

(57) ABSTRACT

A guide device for guiding a medical instrument relative to a patient. The guide device is usable with a first plate including a plurality of apertures. The guide device includes a body, a plurality of outer passageways, and a flexible member. The body is defined by at least one surface that includes a generally proximal portion and a generally distal portion. The plurality of outer passageways extend from the generally proximal portion through the body to the generally distal portion. A flexible member is disposed between each passageway. The flexible member defines an inner passageway and is selectively deformable such that a selected passageway of the plurality of outer passageways or the inner passageway is configured to receive at least a portion of the medical instrument.

### FIELD OF THE INVENTION

The present invention relates generally to targeting cubes used with vacuum-assisted breast biopsy devices for use in breast biopsy procedures conducted with MRI.

### BACKGROUND

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, Positron Emission Mammography (“PEM” guidance), Breast-Specific Gamma Imaging (“BSGI”) guidance or otherwise.

The state of the art technology for conducting a breast biopsy is to use a vacuum-assisted breast biopsy device. A current textbook in this area is “Vacuum-Assisted Breast Biopsy with Mammotome®”, available Nov. 11, 2012, copyright 2013 by Devicor Medical Germany GmBh, published in Germany by Springer Medizin Verlag, Authors: Markus Hahn, Anne Tardivon and Jan Casselman, ISBN 978-3-642-34270-7, http://www.amazon.com/Vacuum-Assisted-Breast-Biopsy-Mammotome-Diagnostic/dp/3642342701?ie=UTF8&keywords=vacuum%20assisted %20breast%20biopsy%20with%20Mammotome&qid=1460663723&ref_=sr_1_1&sr=8−1.

Known biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “Mill Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 1, 2012; U.S. Pat. No. 8,206,316, entitled “Tetherless Biopsy Device with Reusable Portion,” issued on Jun. 26, 2012; U.S. Pat. No. 8,241,226, entitled “Biopsy Device with Rotatable Tissue Sample Holder,” issued on Aug. 14, 2012; U.S. Pat. No. 8,251,916, entitled “Revolving Tissue Sample Holder for Biopsy Device,” issued Aug. 28, 2012; U.S. Pat. No. 8,454,531, entitled “Icon-Based User Interface on Biopsy System Control Module,” published May 21, 2009, issued on Jun. 4, 2013; U.S. Pat. No. 8,532,747, entitled “Biopsy Marker Delivery Device,” issued Sep. 10, 2013; U.S. Pat. No. 8,702,623, entitled “Biopsy Device with Discrete Tissue Chambers,” issued on Apr. 22, 2014; U.S. Pat. No. 8,764,680, entitled “Handheld Biopsy Device with Needle Firing,” issued on Jun. 11, 2014; U.S. Pat. No. 8,801,742, entitled “Needle Assembly and Blade Assembly for Biopsy Device,” issued Aug. 12, 2014; U.S. Pat. No. 8,858,465, entitled “Biopsy Device with Motorized Needle Firing,” issued Oct. 14, 2014; U.S. Pat. No. 8,938,285, entitled “Access Chamber and Markers for Biopsy Device,” issued Jan. 20, 2015; U.S. Pat. No. 9,095,326, entitled “Biopsy System with Vacuum Control Module,” issued Aug. 4, 2015 and U.S. Pat. No. 9,095,326, entitled “Biopsy System with Vacuum Control Module,” issued Aug. 4, 2015. The disclosure of each of the above-cited U.S. Patents is incorporated by reference herein.

Additional known biopsy devices and biopsy system components are disclosed in U.S. Pat. Pub. No. 2006/0074345, entitled “Biopsy Apparatus and Method,” published Apr. 6, 2006 and now abandoned; U.S. Pat. Pub. No. 2008/0214955, entitled “Presentation of Biopsy Sample by Biopsy Device,” published Sep. 4, 2008; U.S. Pat. Pub. No. 2009/0131821, entitled “Graphical User Interface For Biopsy System Control Module,” published May 21, 2009, now abandoned; U.S. Pat. Pub. No. 2010/0152610, entitled “Hand Actuated Tetherless Biopsy Device with Pistol Grip,” published Jun. 17, 2010, now abandoned; U.S. Pat. Pub. No. 2010/0160819, entitled “Biopsy Device with Central Thumbwheel,” published Jun. 24, 2010, now abandoned; U.S. Pat. Pub. No. 2013/0053724, entitled “Biopsy Device Tissue Sample Holder with Bulk Chamber and Pathology Chamber,” published Feb. 28, 2013, will issue on May 3, 2016 as U.S. Pat. No. 9,326,755; U.S. Pat. Pub. No. 2013/0144188, entitled “Biopsy Device With Slide-In Probe,” published Jun. 6, 2013; and U.S. Pat. Pub. No. 2013/0324882, entitled “Control for Biopsy Device,” published Dec. 5, 2013. The disclosure of each of the above-cited U.S. Patent Application Publications, U.S. Non-Provisional Patent Applications, and U.S. Provisional Patent Applications is incorporated by reference herein.

U.S. Pat. Nos. 7,322,990, “Needle Guide For Stereotactic Biopsy”, issued on 29 Jan. 2008 to Suros Surgical Systems, Inc.; U.S. Pat. No. 7,347,829, “Introduction System for Minimally Invasive Surgical Instruments, issued on 25 Mar. 2008 To Suros Surgical Systems, Inc.; U.S. Pat. No. 7,379,769 “Hybrid Imaging Method To Monitor Medical Device Delivery and Patient Support for use in the Method, issued on 27 May 2008 to Sunnybrook Health Sciences Center; U.S. Pat. No. 7,740,593, “Guide Block for Biopsy or Surgical Devices”, issued on 22 Jun. 2010 to Senorx, Inc.; U.S. Pat. No. 7,744,543, “Guide Block for Biopsy or Surgical Devices”, issued on 29 Jun. 2010 to Senorx, Inc.; U.S. Pat. No. 7,970,452, “Open Architecture Imaging Apparatus and Coil System for Magnetic Resonance Imaging”, issued on 28 Jun. 2011 to Hologic, Inc.; U.S. Pat. No. 8,057,432, “Selective Locking Mechanism for an Introducer Device”, issued on 15 Nov. 2011 to Suros Surgical Systems, Inc.; U.S. Pat. No. 8,162,847, “MRI Biopsy Targeting Cube with Snap Corners”, issued on 24 Apr. 2012 to Devicor Medical Products, Inc. ; U.S. Pat. No. 8,162,848, “MRI Biopsy Targeting Cube with Eccentric Lock”, issued on 24 Apr. 2012 to Devicor Medical Products, Inc.; U.S. Pat. No. 8,162,849, “Mill Biopsy Targeting Cube with Gripping Arms”, issued on 24 Apr. 2012 to Devicor Medical Products, Inc.; U.S. Pat. No. 8,167,814, “Biopsy Targeting Cube with Malleable Members”, issued on 1 May 2012 to Devicor Medical Products, Inc.; U.S. Pat. No. 8,197,495, “Biopsy Targeting Cube with Elastomeric Edges,” issued on 12 Jun. 2012 to Devicor Medical Products, Inc.; U.S. Pat. No. 8,241,302, “Biopsy Targeting Cube with Angled Interface”, issued on 14 Aug. 2012 to Devicor Medical Products, Inc.; U.S. Pat. No. 8,366,634, “Biopsy Targeting Cube with Elastomeric Body”, issued on 5 Feb. 2013 to Devicor Medical Products, Inc.; U.S. Pat. No. 8,398,565, “Guide Block for Biopsy or Surgical Devices”, issued on 19 Mar. 2013 to Senorx, Inc.; U.S. Pat. No. 8,554,309, “Localizing Obturator with Site Marking Capability”, issued on 8 Oct. 2013 to Hologic, Inc.; U.S. Pat. No. 8,568,333, “Grid and Rotatable Cube Guide Localization Fixture for Biopsy Device”, issued on 29 Oct. 2013 to Devicor Medical Products, Inc.; U.S. Pat. No. 8,571,632, “Open Architecture Imaging Apparatus and Coil System for Magnetic Resonance Imaging”, issued 29 Oct. 2013 to Hologic, Inc.; U.S. Pat. No. 8,747,331, “Variable Angle Guide Holder for a Biopsy Guide Plug”, issued 10 Jun. 2014 to Hologic, Inc.; U.S. Pat. No. 8,858,537, “Biopsy Targeting Cube with Living Hinges”, issued on 14 Oct. 2014 to Devicor Medical Products, Inc.; U.S. Pat. No. 9,055,926, “Guide Block for Biopsy or Surgical Devices”, issued on 16 Jun. 2015 to SenoRx, Inc.; and US Published Pat. Application No. 2014/0128883 (abandoned January 2016), “Open Architecture Imaging Apparatus and Coil System for Magnetic Resonance Imaging” filed 27 Sep. 2013 by Hologic, Inc., all describe and claim breast biopsy procedures. The disclosure of each of the above-cited U.S. Patent Application Publications, and issued U.S. Patents are all incorporated by reference herein.

European Granted Patents and Published Patent Applications EP1598006B2, “MRI biopsy device localization fixture”, granted 26 Mar. 2008 to Ethicon Endo-Surgery Inc.; EP1598015B2, “MRI biopsy apparatus incorporating a sleeve and multi-function obturator” granted 29 Sep. 2010 to Ethicon Endo-Surgery Inc.; EP1852070B2, “Biopsy cannula adjustable depth stop”, granted 19 Aug. 2009 to Ethicon Endo-Surgery Inc.; EP1859742A1, “MRI biopsy device”, filed 22 May 2007 by Ethicon Endo-Surgery Inc., withdrawn 30 Jan. 2009; EP2111799 A1, “Biopsy cannula adjustable depth stop”, filed 30 Apr. 2007 by Ethicon Endo-Surgery Inc., pending; EP2113204 B2, “PEM and BSGI biopsy devices and methods” , granted 25 Mar. 2015 to Devicor Medical Products, Inc.; EP2229890 A1, “Methods for Imaging” filed Mar. 18, 2010 by Ethicon Endo-Surgery, Inc., deemed to be withdrawn 2011; EP2229908 A1, EP 2263577 A2, EP 2263578 A2, EP 2283772 B1, “MRI Biopsy Device Localization Fixture”, granted 14 Mar. 2012 to Ethicon Endo-Surgery, Inc.; EP 2485650 A1, “MRI Biopsy Targeting Cube with Retention Wiper” EP 2786710 A1, “PEM and BSGI biopsy devices”, filed Apr. 22, 2009 to Ethicon Endo-Surgery, Inc. and WO2014/168214 A1, “RF Coil and Magnetic Resonance Imaging Device”, filed 10 Apr. 2014 to Kabushiki Kaisha Toshiba and Toshiba Medical Systems; all describe and claim breast biopsy procedures.

When the breast biopsy procedure is conducted using MRI, then it is typical to use either a pillar and post type of targeting system to align the breast biopsy device with the target tissue or the medical professional uses a coil containing one or more guide cubes.

In U.S. Pat. Pub. No. 2005/0283069, entitled “MRI Biopsy Device Localization Fixture” published Dec. 22, 2005, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions. Another merely illustrative localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

In U.S. Pat. Pub. No. 2005/0283069, entitled “MRI Biopsy Device Localization Fixture” published Dec. 22, 2005, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions. Another merely illustrative localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

While several systems and methods have been made and used for obtaining a biopsy sample, it is believed that no one prior to the inventor has made or used the invention described in the appended claims.

### SUMMARY OF THE INVENTION

The first aspect of the instant claimed invention is a guide device for guiding a medical instrument relative to a patient, the guide device being usable with a first plate and a second plate, wherein the first plate has a plurality of apertures, wherein the second plate and the first plate are adjustable to secure a portion of the patient, wherein the guide device is configured to be coupled with a selected one of the apertures of the first plate, the guide device comprising: a body defined by at least one surface, wherein the at least one surface comprises a generally proximal portion of the body and a generally distal portion of the body; a plurality of outer passageways defined by at least a portion of the body, wherein the plurality of outer passageways extend from the generally proximal portion through the body to the generally distal portion; and a flexible member disposed between each passageway of the plurality of passageways, wherein the flexible member defines an inner passageway, wherein the flexible member is selectively deformable such that a selected passageway of the plurality of outer passageways or the inner passageway is configured to receive at least a portion of the medical instrument.

The second aspect of the instant claimed invention is a guide device for guiding a medical instrument relative to a patient, the guide device being usable with a first plate and a second plate, wherein the first plate has a plurality of apertures, wherein the second plate and the first plate are adjustable to secure a portion of the patient, wherein the guide device is configured to be coupled with a selected one of the apertures of the first plate, the guide device comprising: a body defined by at least one surface, wherein the at least one surface comprises a generally proximal portion of the body and a generally distal portion of the body; a plurality of outer passageways defined by at least a portion of the body, wherein the plurality of outer passageways extend from the generally proximal portion through the body to the generally distal portion; and a flexible member disposed between each passageway of the plurality of passageways, wherein the flexible member defines an inner passageway, wherein the flexible member is selectively deformable such that a selected passageway of the plurality of outer passageways or the inner passageway is configured to receive at least a portion of the medical instrument; and wherein the plurality of outer passageways together define four corner passageways, wherein the inner passageway is disposed centrally between the four corner passageways.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an MRI breast biopsy system including a control module remotely coupled to a biopsy device, and including a localization fixture with a lateral grid plate used in conjunction with a rotatable cube to position an obturator or a probe of the biopsy device to a desired insertion depth as set by a ring stop;

FIG. 2 depicts a perspective view of a MRI breast coil receiving the localization fixture of FIG. 1;

FIG. 3 depicts a perspective view of the MRI breast biopsy device inserted through the rotatable cube within the cube plate of the localization fixture attached to the breast coil of FIG. 2;

FIG. 4 depicts a perspective view of a two-axis rotatable guide cube of the MRI breast biopsy system of FIG. 1;

FIG. 5 depicts a diagram of nine guide positions achievable by the two-axis rotatable guide cube of FIG. 4;

FIG. 6 depicts a perspective view of a two-axis rotatable guide cube into a lateral grid with the backing of the localization fixture of FIG. 1;

FIG. 7 depicts a perspective view of a obturator and cannula of the MRI breast biopsy system of FIG. 1;

FIG. 8 depicts a perspective exploded view of the obturator and cannula of FIG. 7;

FIG. 9 depicts a perspective view of the obturator and cannula of FIG. 7 with a depth stop device of FIG. 1 inserted through the guide cube and grid plate of FIG. 6;

FIG. 10 depicts a perspective view of an exemplary alternative guide cube for use with the MRI breast biopsy system of FIG. 1;

FIG. 11 depicts a rear elevational view of the guide cube of FIG. 10;

FIG. 12 depicts a side cross-sectional view of the guide cube of FIG. 10, with the cross-section taken along line 12-12 of FIG. 10;

FIG. 13 depicts a perspective view of the guide cube of FIG. 10 inserted into the grid plate of FIG. 6;

FIG. 14 depicts a perspective view of the guide cube of FIG. 10, with the cannula of FIG. 7 inserted through a central opening of the guide cube;

FIG. 15 depicts a perspective view of the guide cube of FIG. 10, with the cannula of FIG. 7 inserted through a corner opening of the guide cube;

FIG. 16 depicts a rear elevational view of the guide cube of FIG. 10, with a flexible member displaced inwardly;

FIG. 17 depicts a perspective view of an exemplary alternative guide device for use with the biopsy system of FIG. 1;

FIG. 18 depicts a rear elevational view of the guide device of FIG. 17;

FIG. 19 depicts a perspective view of the guide device of FIG. 17 inserted into the grid plate of FIG. 6;

FIG. 20 depicts a perspective view of the guide device of FIG. 17, with the cannula of FIG. 7 partially inserted through an opening of the guide device and the grid plate of FIG. 6;

FIG. 21 depicts a perspective view of the guide device of FIG. 17, with the cannula of FIG. 7 fully inserted through opening of FIG. 20 and the grid plate of FIG. 6; and

FIG. 22 depicts a perspective view of the guide device of FIG. 17, with the cannula of FIG. 7 fully inserted through a guide channel of the guide device and the grid plate of FIG. 6.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION OF THE INSTANT CLAIMED INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

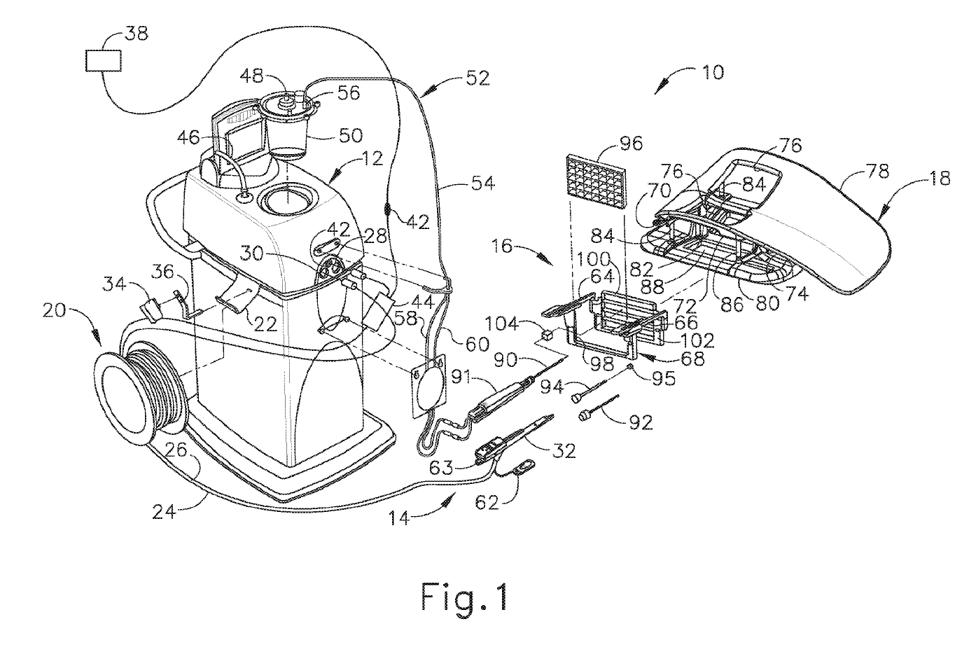

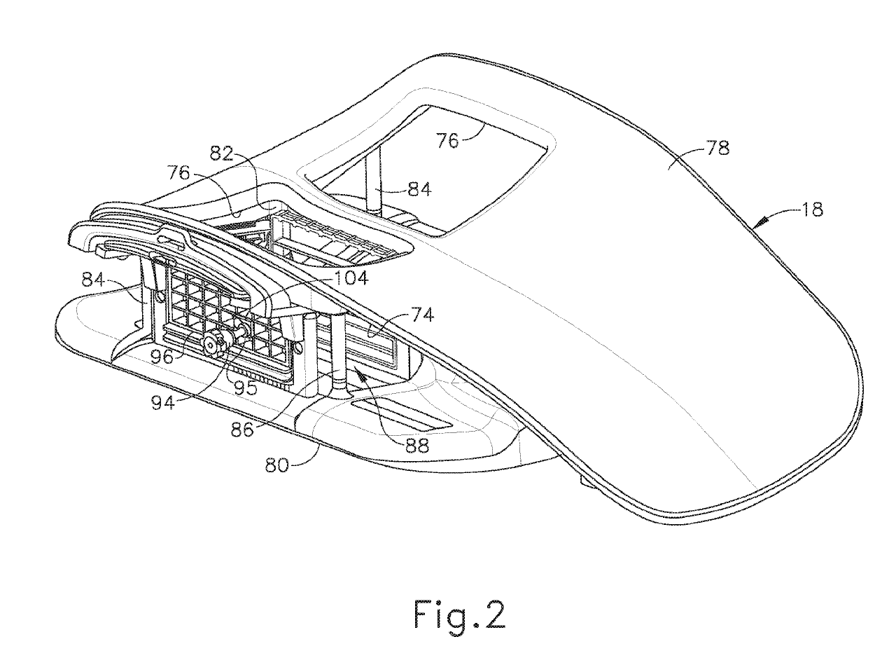

In FIGS. 1-3, MRI compatible breast biopsy system (10) has control module (12) that may be placed outside of a shielded room containing an MRI machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MRI machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MRI machine or other machine. It should be understood that interface lock box (38) is generally movable and/or mountable in multiple positions. Additionally, interface lock box (38) may be moved during transport of control module (12). In-line enclosure (44) may register tether (40), electrical cable (24) and mechanical cable (26) to their respective ports (42, 28, 30) on control module (12). Although lockout port (42) is shown as being coupled to in-line enclosure (44), it should be understood that in other examples, lockout port (42) may be located in other positions. For instance, in some examples lockout port (42) is located on the rear of control module (12) and is therefore not associated with in-line enclosure (44).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pub. No. 2008/0228103, entitled “Vacuum Timing Algorithm for Biopsy Device,” published Sep. 18, 2008, the disclosure of which is incorporated by reference herein. As another merely illustrative example, control module (12) may instead be configured and operable in accordance with the teachings of U.S. Pat. No. 8,328,732, entitled “Control Module Interface for MRI Biopsy Device,” issued Dec. 11, 2012, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

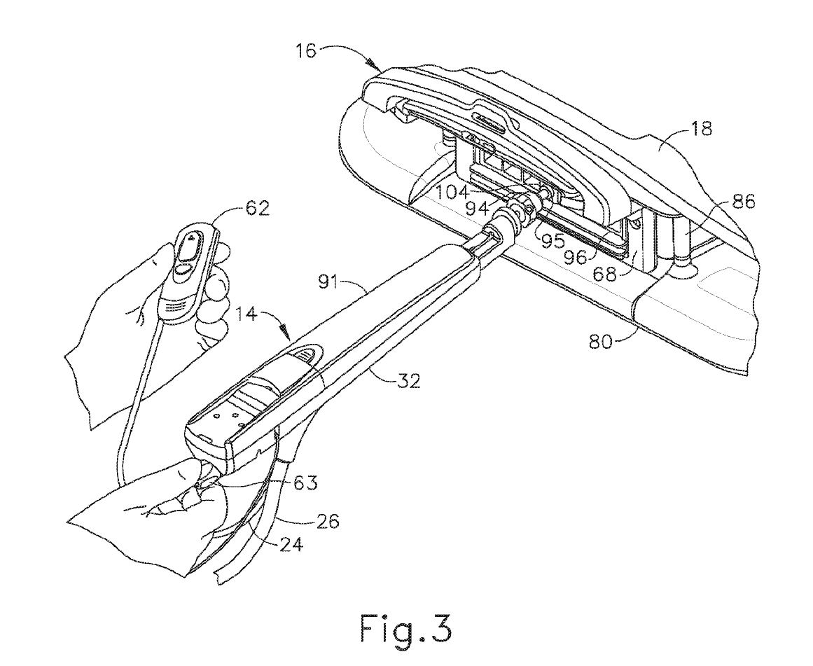

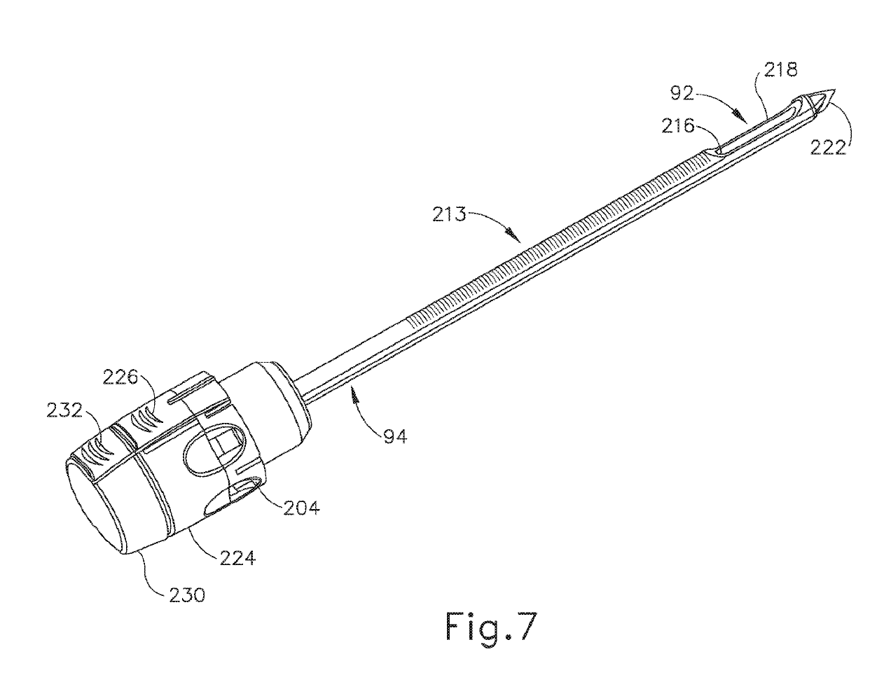

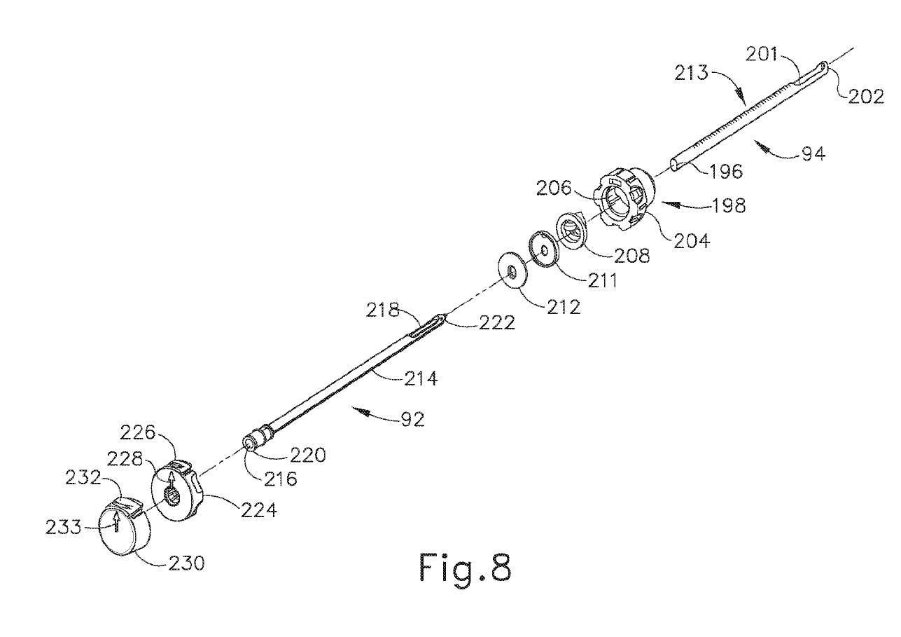

In the present example, a targeting set (89) comprising cannula (94) and obturator (92) is associated with probe (91). In particular, and as shown in FIGS. 7, 8, and 9, obturator (92) is slid into cannula (94) and the combination is guided through guide cube (104) to the biopsy site within the breast tissue. As shown in FIG. 3, obturator (92) is then withdrawn from cannula (94), then needle (90) of probe (91) is inserted in cannula (94), and then biopsy device (14) is operated to acquire one or more tissue samples from the breast via needle (90).

Cannula (94) of the present example is proximally attached to cylindrical hub (198) and cannula (94) includes lumen (196) and lateral aperture (201) proximate to open distal end (202). Cylindrical hub (198) has exteriorly presented thumbwheel (204) for rotating lateral aperture (201). Cylindrical hub (198) has interior recess (206) that encompasses duckbill seal (208), wiper seal (211) and seal retainer (212) to provide a fluid seal when lumen (196) is empty and for sealing to inserted obturator (92). Longitudinally spaced measurement indicia (213) along an outer surface of cannula (94) visually, and perhaps physically, provide a means to locate depth stop device (95) of FIG. 1.

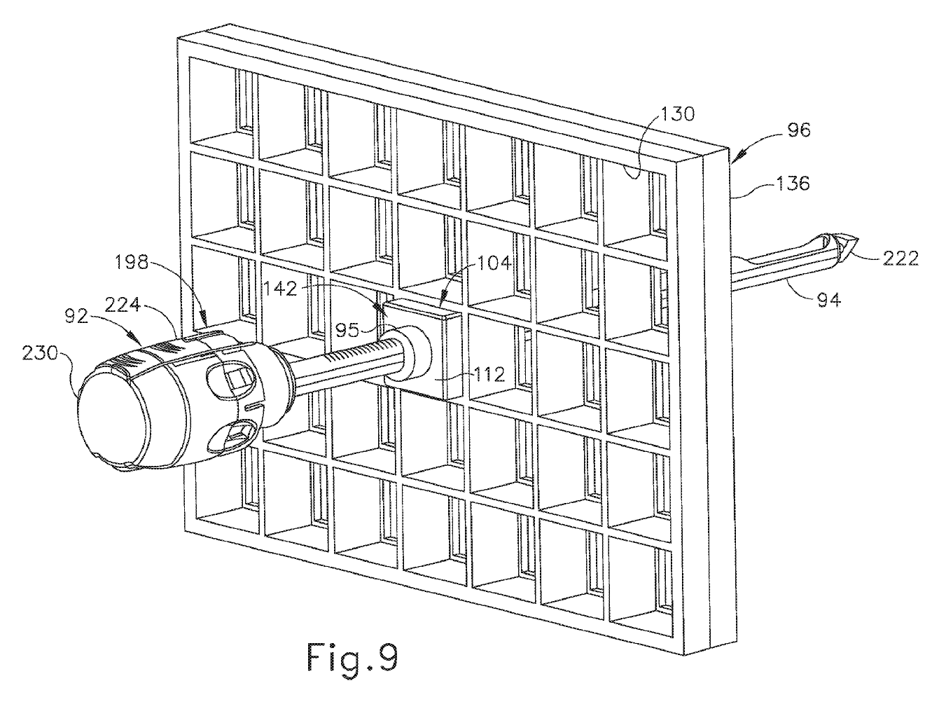

Obturator (92) of the present example incorporates a number of components with corresponding features. Shaft (214) includes fluid lumen (216) that communicates between imageable side notch (218) and proximal port (220). Shaft (214) is longitudinally sized such that piercing tip (222) extends out of distal end (202) of cannula (94). Obturator thumbwheel cap (224) encompasses proximal port (220) and includes locking feature (226), which includes visible angle indicator (228), that engages cannula thumbwheel (204) to ensure that imageable side notch (218) is registered to lateral aperture (201) in cannula (94). Obturator seal cap (230) may be engaged proximally into obturator thumbwheel cap (224) to close fluid lumen (216). Obturator seal cap (230) of the present example includes locking or locating feature (232) that includes visible angle indicator (233) that corresponds with visible angle indicator (228) on obturator thumbwheel cap (224), which may be fashioned from either a rigid, soft, or elastomeric material. In FIG. 9, guide cube (104) has guided obturator (92) and cannula (94) through grid plate (96).

While obturator (92) of the present example is hollow, it should be understood that obturator (92) may alternatively have a substantially solid interior, such that obturator (92) does not define an interior lumen. In addition, obturator (92) may lack side notch (218) in some versions. Other suitable components, features, configurations, functionalities, operability, etc. for an obturator (92) will be apparent to those of ordinary skill in the art in view of the teachings herein. Likewise, cannula (94) may be varied in a number of ways. For instance, in some other versions, cannula (94) has a closed distal end (202). As another merely illustrative example, cannula (94) may have a closed piercing tip (222) instead of obturator (92) having piercing tip (222). In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop (95). Depth stop may be of any suitable configuration that is operable to prevent cannula (94) and obturator (92) (or needle (90)) from being inserted further than desired. For instance, depth stop (95) may be positioned on the exterior of cannula (94) (or needle (90)), and may be configured to restrict the extent to which cannula (94) is inserted into a guide cube. It should be understood that such restriction by depth stop (95) may further provide a limit on the depth to which the combination of cannula (94) and obturator (92) (or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stops (95) that may be used with biopsy system (10) are described in U.S. Pub. No. 2007/0255168, entitled “Grid and Rotatable Cube Guide Localization Fixture for Biopsy Device,” published Nov. 1, 2007, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with lateral aperture (201) of cannula (94) when needle (90) is inserted into lumen (196) of cannula (94). Probe (91) of the present example further comprises a rotating and translating cutter (not shown), which is driven by components in holster (32), and which is operable to sever tissue protruding through lateral aperture (201) of cannula (94) and the lateral aperture of needle (90). Severed tissue samples may be retrieved from biopsy device (14) in any suitable fashion.

It should be understood that although biopsy system (10) is discussed above as utilizing disposable probe assembly (91), other suitable probe assemblies and biopsy device assemblies may be utilized. By way of example only, biopsy device (14) may be configured in accordance with at least some of the teachings of U.S. Pat. No. 8,206,316, entitled “Tetherless Biopsy Device with Reusable Portion,” issued Jun. 26, 2012, the disclosure of which is incorporated by reference herein; U.S. Pat. No. 8,277,394, entitled “Multi-Button Biopsy Device,” issued Oct. 2, 2012, the disclosure of which is incorporated by reference herein; and/or U.S. Pub. No. 2012/0065542, entitled “Biopsy Device Tissue Sample Holder with Removable Tray,” published Mar. 15, 2012, the disclosure of which is incorporated by reference herein. In still other examples, biopsy device (14) may be configured in accordance with at least some of the teachings of U.S. Pub. No. 2010/0160824, the disclosure of which is incorporated by reference herein; U.S. Pat. Pub. No. 2013/0144188, entitled “Biopsy Device with Slide-In Probe,” published Jun. 6, 2013, the disclosure of which is incorporated by reference herein; U.S. Pat. Pub. No. 2013/0324882, entitled “Control for Biopsy Device,” published Dec. 5, 2013, the disclosure of which is incorporated by reference herein; U.S. Pat. Pub. No. 2014/0039343, entitled “Biopsy System,” published Feb. 6, 2014, the disclosure of which is incorporated by reference herein; and/or U.S. patent application Ser. No. 14/469,761, entitled “Tissue Collection Assembly for Biopsy Device,” filed Aug. 27, 2014, the disclosure of which is incorporated by reference herein.

Still other suitable forms of biopsy devices that may be used in conjunction with the various alternative components of system (10) as described herein will be apparent to those of ordinary skill in the art.

In some versions, a guide cube may comprise a body defined by one or more edges and faces. The body may include one or more guide holes or other types of passages that extend between faces of the guide cube and that may be used to guide an instrument such as a biopsy device (14) or a portion of a biopsy device (14) (e.g., needle (90) of biopsy device (14), a combination of cannula (94) and obturator (92), etc.). Guide cubes may be rotatable about one, two, or three axes to position the one or more guide holes or passages of the guide cube into a desired position.

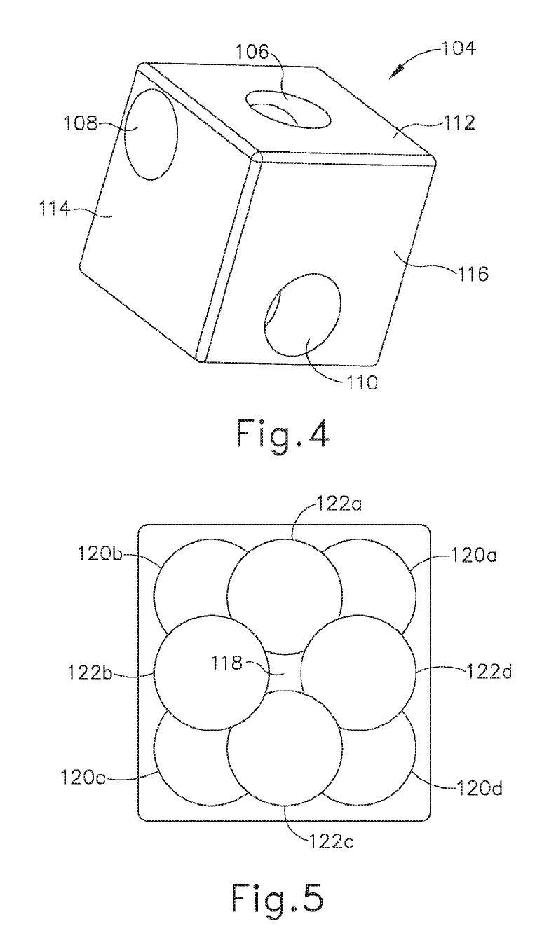

In FIG. 4, guide cube (104) includes a central guide hole (106), a corner guide hole (108), and an off-center guide hole (110) that pass orthogonally to one another between respective opposite pairs of faces (112, 114, 116). By selectively rotating guide cube (104) in two axis, one of pairs of faces (112, 114, 116) may be proximally aligned to an unturned position and then selected proximal face (112, 114, 116) optionally rotated a quarter turn, half turn, or three quarter turn. Thereby, one of nine guide positions (118) (i.e., using central guide hole (106)), (120a-120d) (i.e., corner guide hole (108)), (122a-122d) (i.e., using off-center guide hole (110)) may be proximally exposed as depicted in FIG. 5.

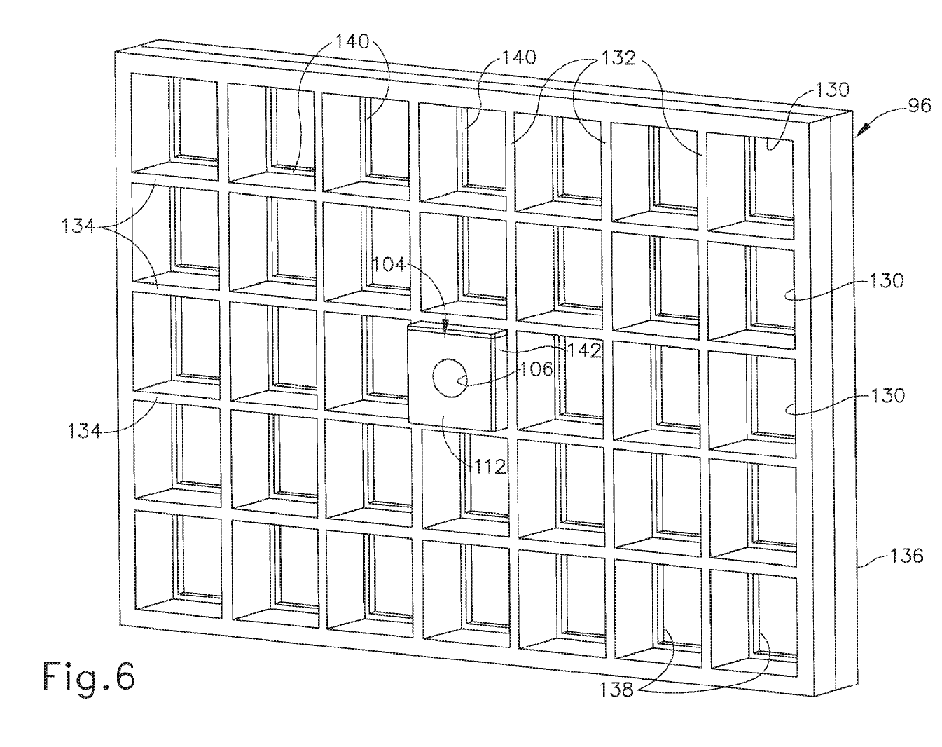

In FIG. 6, two-axis rotatable guide cube (104) is sized for insertion from a proximal side into one of a plurality of square recesses (130) in grid plate (96), which are formed by intersecting vertical bars (132) and horizontal bars (134). Guide cube (104) is prevented from passing through grid plate (96) by backing substrate (136) attached to a front face of grid plate (96). Backing substrate (136) includes respective square opening (138) centered within each square recess (130), forming lip (140) sufficient to capture the front face of guide cube (104), but not so large as to obstruct guide holes (104, 106, 108). The depth of square recesses (130) is less than guide cube (104), thereby exposing a proximal portion (142) of guide cube (104) for seizing and extraction from grid plate (96). It will be appreciated by those of ordinary skill in the art based on the teachings herein that backing substrate (136) of grid plate (96) may be omitted altogether in some versions. In some such versions without backing substrate (136) other features of a guide cube, as will be discussed in more detail below, may be used to securely and removably fit a guide cube within a grid plate. However, such other features may also be used in combination with a grid plate having backing substrate (136), such as grid plate (96), instead of partially or wholly omitting backing substrate (136).

In some other versions, guide cube (104) is replaced with an alternative guide cube or other guide structure that is configured and operable in accordance with at least some of the teachings of U.S. patent application Ser. No. 14/335,051, entitled “Biopsy Device Targeting Features,” filed Jul. 18, 2014, the disclosure of which is incorporated by reference herein.

In some examples obturator (92) and cannula (94) discussed above may be usable with a different guide device, instead of depth stop device (104). In addition or in the alternative, guide cube (104) may be usable in conjunction with certain adaptor features. Such alternative guide devices may be desirable to generally improve the usability and or functionality of obturator (92) and/or cannula (94). For instance, in some examples guide devices may include features and/or components that permit guide devices to receive obturator (92) and/or cannula (94) at multiple positions across a particular face of the guide devices. Such features may permit this functionality without necessitating repositioning of guide devices relative to a patient. Various examples of suitable guide devices will be described in greater detail below; while other examples will be apparent to those of ordinary skill in the art according to the teachings herein. It should be understood that the guide device examples described below may function substantially similar to guide cube (104) and may be readily usable with obturator (92) and cannula (94) described above. In particular, the guide device examples described below may be used to assist in biopsy device needle targeting within a patient's breast using MRI guidance. It should also be understood that the guide device examples discussed below may be used with any of the biopsy devices discussed above or otherwise disclosed herein.

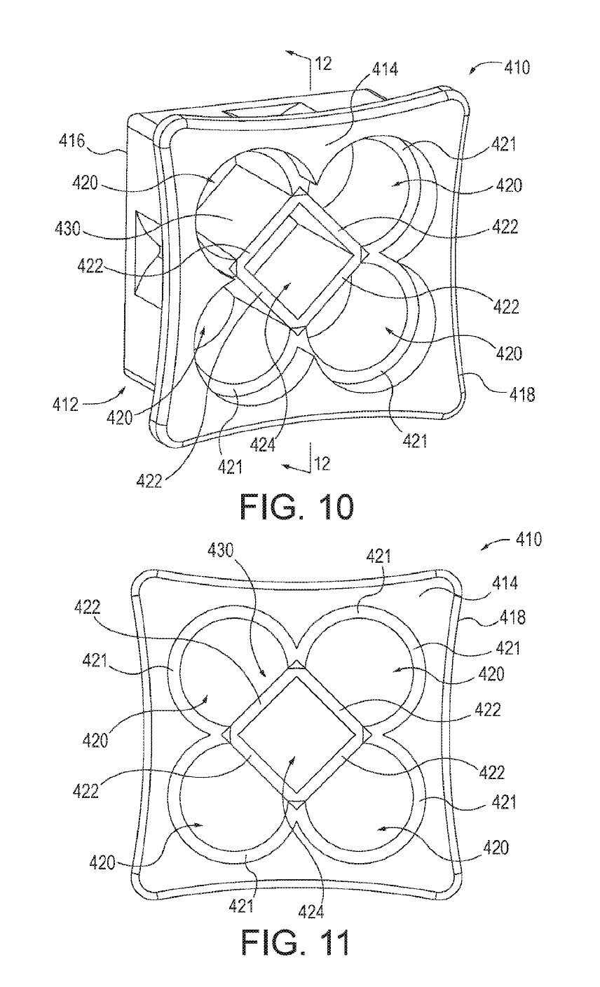

FIG. 10 shows an exemplary alternative guide cube (410) that may be usable with obturator (92) and/or cannula (94), described above, in conjunction with or in lieu of guide cube (104), described above. Unless otherwise described herein, it should be understood that guide cube (410) is substantially similar to guide cube (104). For instance, guide cube (410) comprises a plurality of openings (420, 424) that are configured to receive obturator (92) and/or cannula (94) as described above with respect to holes (106, 108, 110) of guide cube (104). However, as will be described in greater detail below, unlike guide cube (104), openings (420, 424) are generally positioned to permit repositioning of obturator (92) and/or cannula (94) without repositioning guide cube (410).

Guide cube (410) comprises a generally cube shaped body (412), which includes a proximal face (414) and a distal face (416). Openings (420, 424) generally extend between proximal and distal faces (414, 416), as will be described in greater detail below. Proximal face (414) includes a lip feature (418) extending outwardly from body (412) around the perimeter of proximal face (414). Body (412) comprises a generally rigid material.

Guide cube (410) further includes a flexible member (430) extending from proximal face (414) to distal face (416). Flexible member (430) of the present example comprises a generally flexible material. Although body (412) is described herein as being generally rigid and flexible member (430) is described herein as being generally flexible, it should be understood that such terms are used herein in a relative sense. For instance, in some examples body (412) and flexible member (430) are comprised of the same material, but with each component being configured with different characteristics. Such characteristics correspond to the relative rigidity and flexibility between body (412) and flexible member (430). In other examples, body (412) may have some flexible characteristics, but still be rigid in comparison to flexible member (430). Similarly, flexible member (430) may have some rigid characteristics, but still be flexible in comparison to body (412). In some examples, the relative relationship between body (412) and flexible member (430) is characterized by durometer. By way of example only, in some examples body (412) comprises a durometer of between 50 and 75 (D scale), while flexible member (430) comprises a durometer of between 15 and 30 (OO scale). Of course, any other suitable relative relationship between body (412) and flexible member (430) may be used as will be apparent to those of ordinary skill in the art.

In addition, it should be understood that in some examples flexible member (430) includes elastomeric properties. As will be understood, such elastomeric properties provide a close fit between cannula (94) and flexible member (430) to generate friction between the two. In some examples flexible member (430) is configured to provide a sufficient amount of such friction to resist movement of cannula (94) relative to flexible member (430). In examples providing such an amount of friction, flexible member (430) is operable to prevent cannula (94) from inadvertently backing out of guide cube (410).

Flexible member (430) forms at least a portion of the wall of each opening (420, 424), thereby defining the particular shape of a given opening (420, 424). As is best seen in FIGS. 10 and 11, when flexible member (430) is in a relaxed position, flexible member (430) is biased toward any shape that fits within the cube configuration. In an embodiment the shape may be generally rectangular. In another embodiment the shape may be a square cross-sectional shape. In other embodiments, flexible member (430) may be biased toward numerous alternative shapes. For instance, in some embodiments flexible member (430) comprises a generally round shape. In other embodiments, flexible member (430) comprises a shape having a cross-section with a plurality of semi-circles joined together. In still other embodiments, flexible member (430) comprises a generally cylindrical shape. In yet other examples, any other suitable shape may be used as will be apparent to those of ordinary skill in the art in view of the teachings herein.

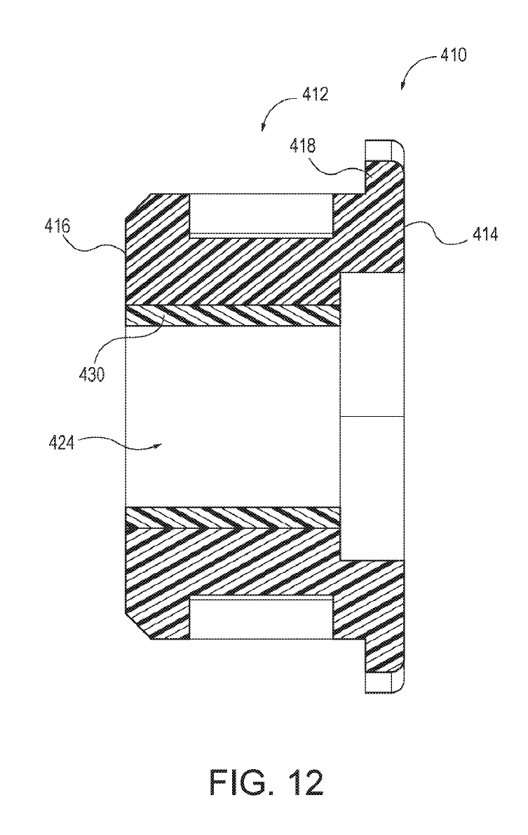

As can be seen in FIG. 12, flexible member (430) is a discrete component relative to body (412). Because of this, it should be understood that flexible member (430) of the present example is secured to body (412). Flexible member (430) may be secured to body (412) by any suitable means such as adhesive bonding, mechanical fastening features, ultrasonic welding, or any other fastening means as will be apparent to those of ordinary skill in the art in view of the teachings herein. Additionally, although not shown, it should be understood that in some examples flexible member (430) is integral with body (412). It should be understood that in examples where flexible member (430) is of integral construction with body (412), flexible member (430) is still configured to be generally flexible relative to body (412) as described above.

As is best seen in FIGS. 10 and 11, openings (420, 424) extend through body (412) from proximal face (414) to distal face (416). In particular, guide cube (410) includes a plurality of corner openings (420) and a single central opening (424). In the present example, all openings (420, 424) extend distally though guide cube (410) parallel to each other and normal to proximal and distal faces (414, 416). Although not shown, it should be understood that in other examples, openings (420, 424) extend at varying angles relative to each other. Moreover, in other examples one or more openings (420, 424) extend between other faces of guide cube (410) besides proximal and distal faces (414, 416).

In the present example, guide cube (410) includes four corner openings (420) disposed in each corner of proximal and distal faces (414, 416). In other examples, any other suitable number of corner openings (420) may be used. The wall of each corner opening (420) is at least partially defined by body (412). Additionally, the wall of each corner opening (420) is also at least partially defined by flexible member (430). In particular, body (412) defines a generally rigid, generally semi-cylindrical first wall portion (421) for each corner opening (420). A second, flexible, wall portion (422) is defined for each corner opening (420) by flexible member (430). As will be understood, this combination permits each opening (420, 424) to occupy a common space at independent points in time.

The wall of central opening (424) is entirely defined by flexible member (430). Thus, the wall of central opening (424) is generally flexible, particularly where flexible member (430) is adjacent to a given corner opening (420). It should be understood that where flexible member (430) attaches to body (412), the wall of central opening (424) is relatively rigid. This is because the rigid nature of body (412) will locally resist movement of flexible member (430). As can best be seen in FIG. 12, flexible member (430) is generally recessed relative to proximal face (414) of guide cube (410). Although not shown, it should be understood that in some examples flexible member (430) can be configured such that the recess expands or contracts in response to receiving cannula (94) therein.

It should be understood that grid plate (96) described above generally is configured with fixed dimensions from grid plate (96) to grid plate (96). As a consequence, faces (414, 416) of guide cube (410) have a relatively fixed area. With the area of a given face (414, 416) fixed, the overlapping configuration of openings (420, 424) permits a greater number of openings (420, 424) relative to guide cubes of similar size, but with non-overlapping configurations. However, it should be understood that flexible member (430) permits such an overlap while still maintaining each opening (420, 424) as a discrete opening (420, 424).

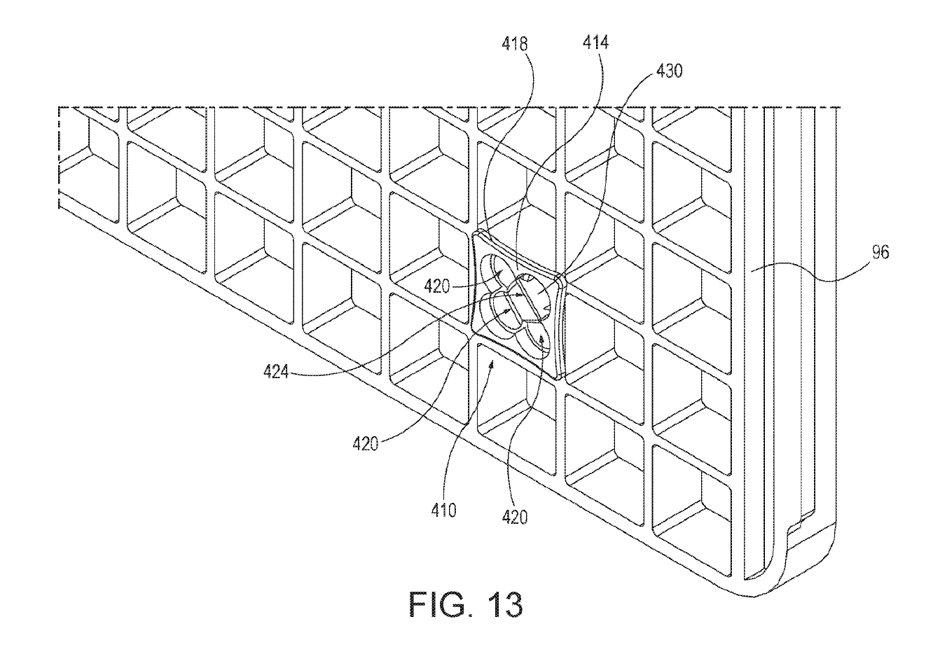

FIGS. 13-16 show an exemplary use of guide cube (410). As can be seen in FIG. 13, guide cube (410) is initially positioned into grid plate (96) as similarly described above with respect to guide cube (104). Lip feature (418) of guide cube (410) prevents over insertion of guide cube (410) by abutting grid plate (96). Once guide cube (410) is positioned within grid plate (96), an operator may readily use obturator (92) and/or cannula (94) in conjunction with guide cube (410).

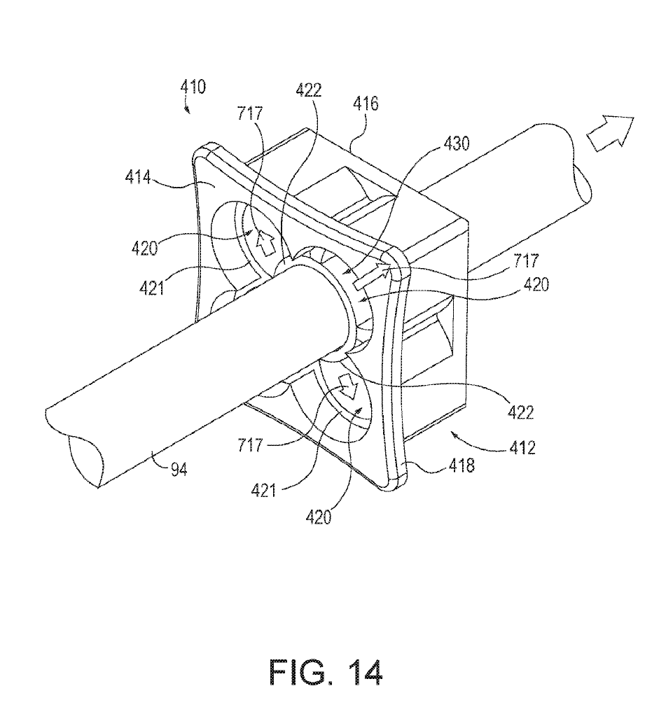

As can be seen in FIG. 14, cannula (94) may be inserted into central opening (424) of guide cube (410) when flexible member (430) is in the relaxed position. As described above, when flexible member (430) is in the relaxed position, flexible member (430) is generally biased to a rectangular or square cross-sectional shape. This shape slightly circumscribes the outer diameter of cannula (94). Thus, as cannula (94) is inserted into central opening (424), at least a portion of flexible member (430) moves outwardly to receive cannula (94). This outward movement, or expansion movement, is depicted in FIG. 14 by arrows (717). It should be understood that the particular amount of movement of flexible member (430) and the particular portion of flexible member (430) that moves is at least partially determined by the shape of flexible member (430). For instance, as can be seen in FIG. 14, the relatively straight walls of flexible member (430) become rounded as cannula (94) is inserted. However, it should be understood that in examples where flexible member (430) comprises alterative shapes, movement of flexible member (430) may be correspondingly altered or even eliminated (e.g., cylindrical flexible member (430)).

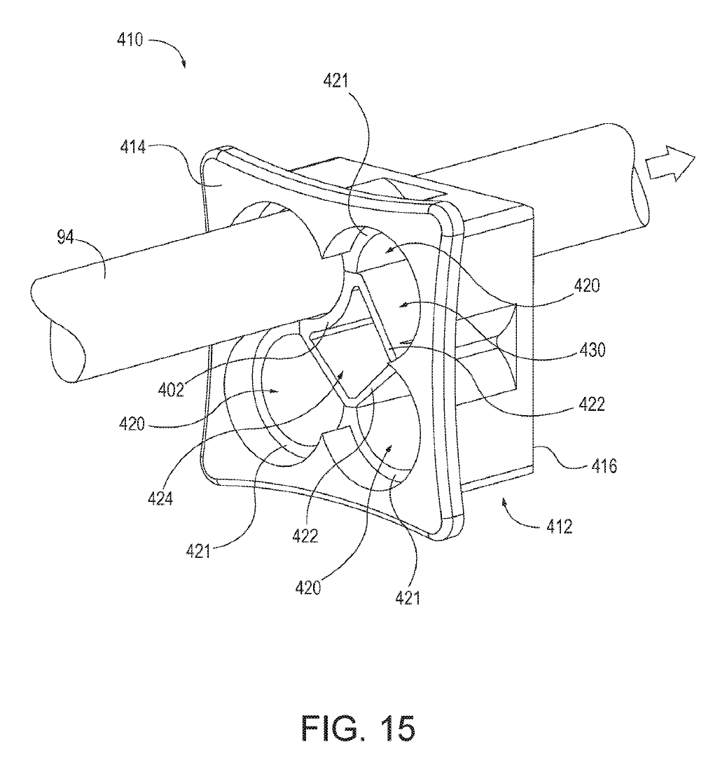

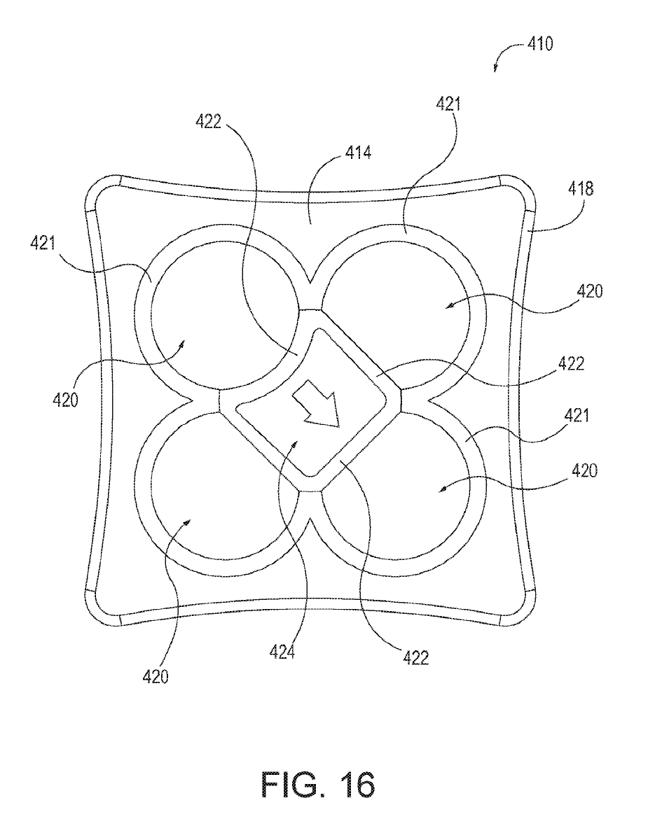

In some instances it may be desirable to change the position of cannula (94). Alternatively, in some uses of guide cube (410) an operator may desire to initially insert cannula (94) into any one of the plurality of corner openings (420). FIG. 15 shows cannula (94) inserted into a corner opening (420) of guide cube (410). As can be seen, when cannula (94) is inserted into corner opening (420) of guide cube (410), at least a portion of flexible member (430) is displaced inwardly. In particular, as is best seen in FIG. 16, insertion of cannula (94) into corner opening (420) will cause the outer wall of cannula (94) to act upon flexible member (430). As cannula (94) acts on flexible member (430), flexible member (430) flexes to conform to the shape of cannula (94). Thus, second wall portion (422) of the wall of corner opening (420) transitions to define a generally circular cross-section with first wall portion (421) of the wall of corner opening (420).

Although cannula (94) is shown in FIG. 15 as being inserted into one particular corner opening (420), it should be understood that in other uses cannula (94) is inserted into any other corner opening (420) as similarly described above. It should also be understood that when moving cannula (94) from one corner opening (420) to another corner opening (420) or to central opening (424), guide cube (410) remains in a generally fixed position relative to grid plate (96). Accordingly, guide cube (410) does not require repositioning to achieve alternative cannula (94) positions.

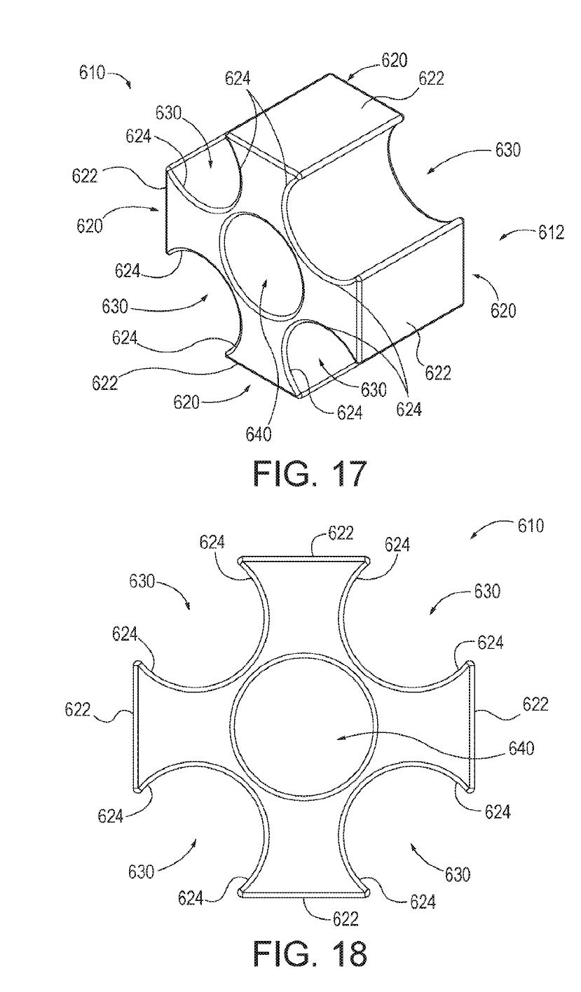

FIGS. 17 and 18 show an exemplary alternative guide device (610) that may be used in conjunction, with or in lieu of, guide cube (104) for targeting obturator (92) and/or cannula (94). Guide device (610) comprises a generally cross-shaped body (612). In particular, body (612) includes four outwardly extending cross members (620). Each cross member (620) is generally oriented at a 90 degree angle relative to another cross member (620) such that cross members (620) together form the legs of the cross shape. Each cross member (620) is generally identical in shape, although individual cross members (620) have various shapes in other examples.

Each particular cross member (620) is also laterally symmetrical widening as the cross member (620) extends outwardly. In particular, each cross member includes an engagement portion (622). Engagement portion (622) is generally flat and is configured to engage a portion of grid plate (96), as will be described in greater detail below. Each engagement portion (622) has a lateral width that is wider than the width of its corresponding cross member (620) at the cross member's (620) base. This change in width defines a curved portion (624) on either lateral side of each cross member (620), which extends longitudinally through the length of body.

Four guide channels (630) are defined by body (612). In particular, for each guide channel (620), a curved portion (624) of two adjacent cross members (620) combine to define a single guide channel (630). Guide channels (630) correspond to the curvature of cannula (94) such that each guide channel (630) is configured to receive cannula (94). However, guide channels (630) are not configured to fully encompass cannula (94) when cannula (94) is received therein. As will be described in greater detail below, guide channels (630) are configured to be usable in conjunction with gird plate (96) to guide cannula (94) relative to a patient.

Body (612) further defines an opening (640) disposed at the intersection of all cross members (620). In other words, opening (640) is generally located at the center of guide device (640). Opening (640) extends longitudinally through body (612) along a path that is generally parallel to guide channels (630). Opening (640) comprises a generally cylindrical shape such that opening (640) is configured to receive cannula (94), as will be described in greater detail below. Although opening (640) is shown as being oriented along a path that is generally parallel to guide channels (630), it should be understood that in other examples opening (640) may be oriented at an angle relative to guide channels (630). Similarly, guide channels (630) may be oriented at an angle relative to each other rather than extending along relatively parallel paths.

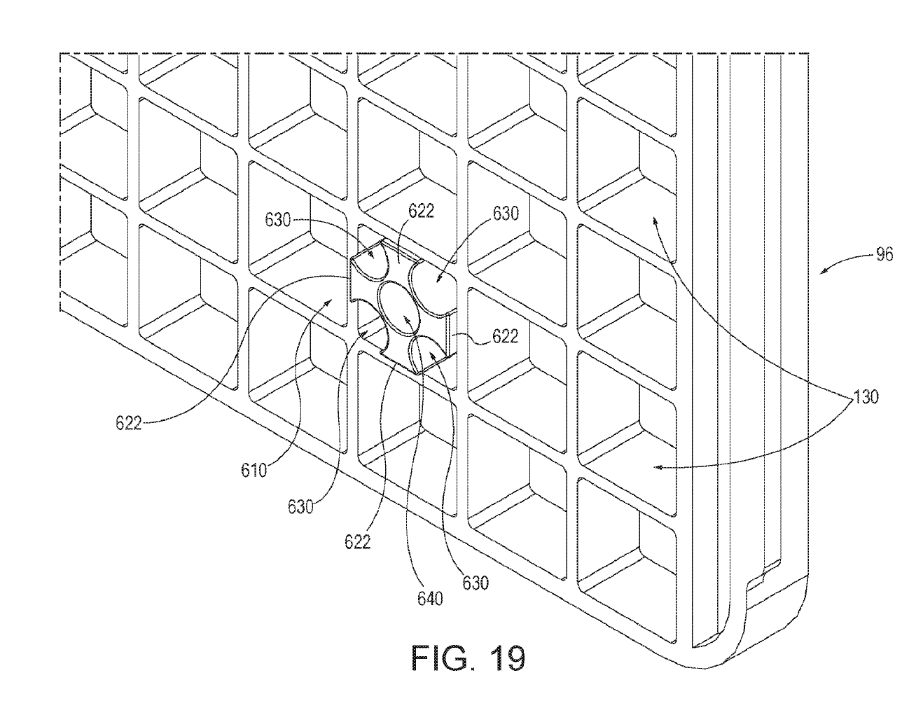

FIGS. 19-22 show an exemplary use of guide device (610). As can be seen in FIG. 19, guide device (610) may be initially inserted by an operator into a particular square recess (130) of grid plate (96). Once guide device (610) is inserted therein, each guide channel (630) forms a cavity that may fully encompass cannula (94) with the particular square recess (130). Additionally, engagement portions (622) of each cross member (620) engage a corresponding wall of the particular square recess (130). In some examples, each cross member (620) may be slightly oversized relative to square recesses (130) such that there is a compression fit between engagement portions (622) and the walls of the particular square recess (130). Of course, such a feature is entirely optional and in some examples guide device (610) may freely slide into the particular square recess (130).

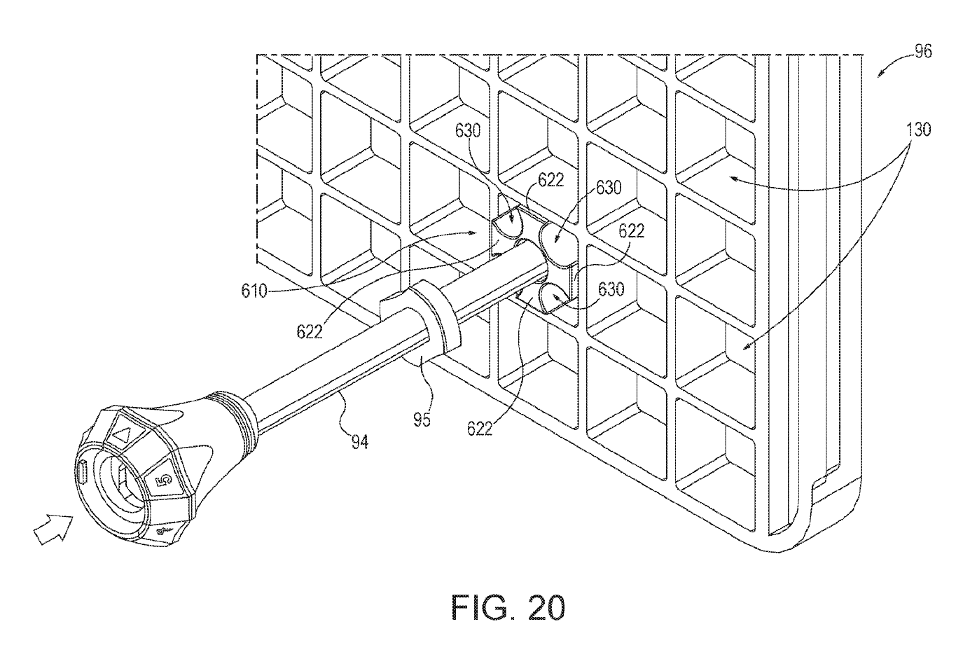

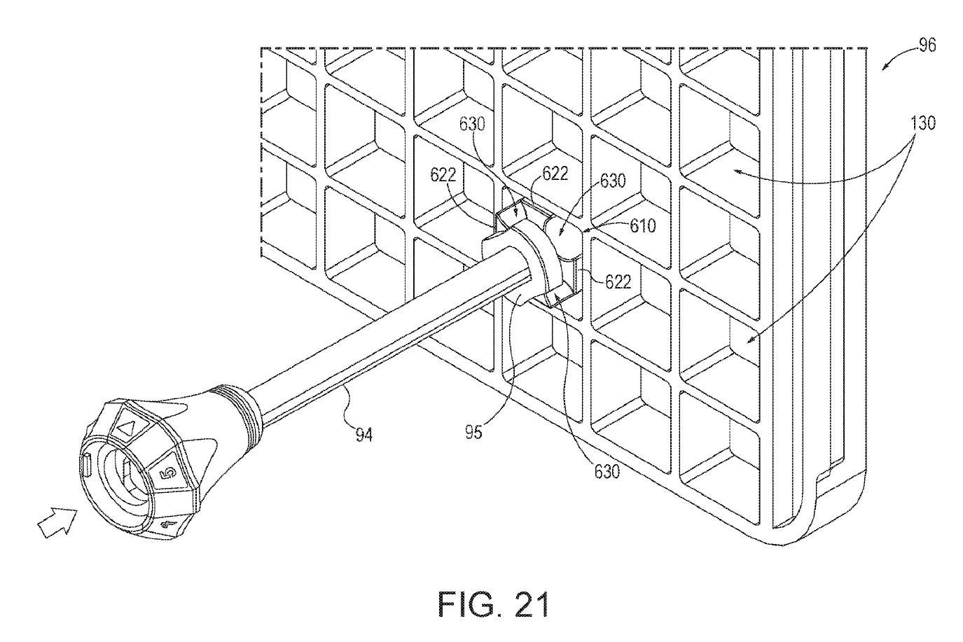

Once guide device (610) is inserted into grid plate (96), cannula (94) may be guided relative to a patient using guide device (610). Any one guide channel (630) or opening (640) may be used to guide cannula (94). For instance, as can be seen in FIG. 20, cannula (94) is initially inserted into opening (640). Cannula (94) may be inserted into guide device (610) until z-stop device (95) arrests further longitudinal movement of cannula (94) through contact with guide device (610), as can be seen in FIG. 21.

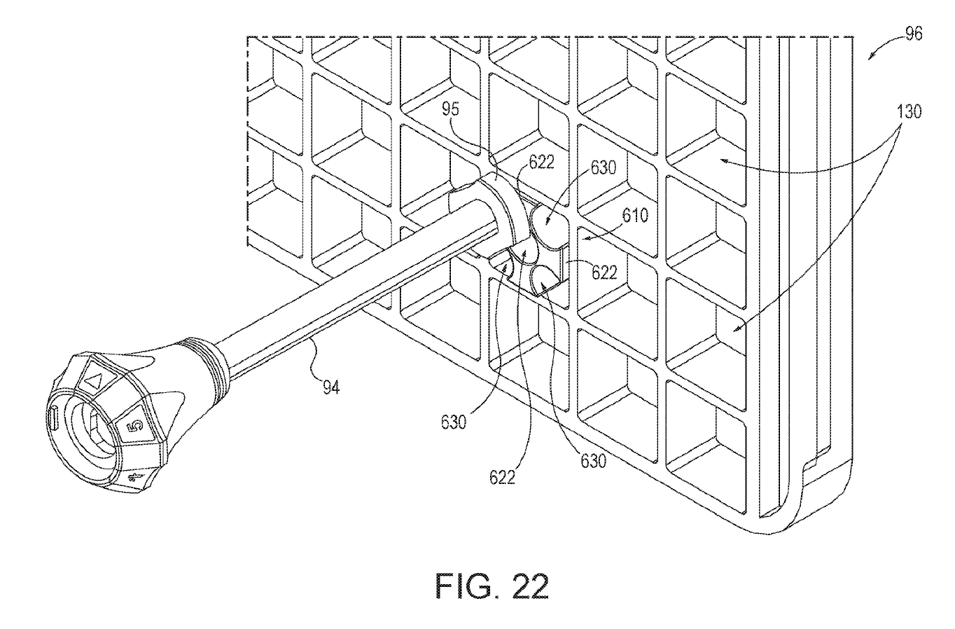

Once cannula (94) is positioned within opening (640), an operator may desire to reposition cannula (94) relative to a patient. Alternatively, prior to insertion of cannula (94) an operator may merely desire to initially position cannula (94) at a different position relative to a patient than the one provided by opening (640). Alternative positions may be accessed by inserting cannula (94) into any one of the spaces defined by each guide channel (630) and square recess (130) of grid plate (96). As can be seen in FIG. 22, cannula (94) is received by a particular guide channel (630) of guide device (610). Once received therein, cannula (94) is held in position by the combination of the particular guide channel (630) and a corresponding sidewall of the square recess (130) of grid plate (96). Thus, guide device (610) and grid plate (96) work cooperatively to maintain the position of cannula (630) relative to a patient. While cannula (94) is shown herein as being received within a particular guide channel (630), it should be understood that cannula (94) may alternatively be received in any one of the guide channels (630).

The present invention has been disclosed with respect to an MRI breast biopsy device. However, the various features and components disclosed in the figures may be employed in devices useful with radioisotope applications, as in PEM, BSGI, and other imaging methods that may employ a radioisotope or other radiation source in connection with imaging a biopsy procedure.

Embodiments of the devices disclosed herein are generally designed to be disposed of after a single use, but could be designed to be used multiple times. After forming the marker, and inserting the marker into the deployer, the biopsy device can be sterilized. The device can be placed in a package, such as plastic or TYVEK bag.

The packaged biopsy device may then be placed in a field of radiation such as gamma radiation, x-rays, or high-energy electrons to sterilize the device and packaging. A device may also be sterilized using any other technique known in the art, including but not limited to beta or gamma radiation, ethylene oxide, or steam

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. A guide device for guiding a medical instrument relative to a patient, the guide device being usable with a first plate and a second plate, wherein the first plate has a plurality of apertures, wherein the second plate and the first plate are adjustable to secure a portion of the patient, wherein the guide device is configured to be coupled with a selected one of the apertures of the first plate, the guide device comprising:
(a) a body defined by at least one surface, wherein the at least one surface comprises a generally proximal portion of the body and a generally distal portion of the body;
(b) a plurality of outer passageways defined by at least a portion of the body, wherein the plurality of outer passageways extend from the generally proximal portion through the body to the generally distal portion; and
(c) a flexible member disposed between each passageway of the plurality of passageways, wherein the flexible member defines an inner passageway, wherein the flexible member is selectively deformable such that a selected passageway of the plurality of outer passageways or the inner passageway is configured to receive at least a portion of the medical instrument.

2. The guide device of claim 1, wherein at least a portion the flexible member is fixedly secured to an inner surface of the body.

3. The guide device of claim 2, wherein the flexible member is fixedly secured to the inner surface of the body at four contact points.

4. The guide device of claim 3, wherein the flexible member is relatively rigid at each contact point of the four contact points.

5. The guide device of claim 1, wherein the flexible member is integral with at least a portion of the body.

6. The guide device of claim 1, wherein the guide device is a guide cube.

7. The guide device of claim 1, wherein the shape of the flexible member defines a square-shaped cross-section.

8. The guide device of claim 1, wherein the shape of the flexible member defines a circle-shaped cross-section.

9. A guide device for guiding a medical instrument relative to a patient, the guide device being usable with a first plate and a second plate, wherein the first plate has a plurality of apertures, wherein the second plate and the first plate are adjustable to secure a portion of the patient, wherein the guide device is configured to be coupled with a selected one of the apertures of the first plate, the guide device comprising:
(a) a body defined by at least one surface, wherein the at least one surface comprises a generally proximal portion of the body and a generally distal portion of the body;
(b) a plurality of outer passageways defined by at least a portion of the body, wherein the plurality of outer passageways extend from the generally proximal portion through the body to the generally distal portion; and
(c) a flexible member disposed between each passageway of the plurality of passageways, wherein the flexible member defines an inner passageway, wherein the flexible member is selectively deformable such that a selected passageway of the plurality of outer passageways or the inner passageway is configured to receive at least a portion of the medical instrument; and
wherein the plurality of outer passageways together define four corner passageways, wherein the inner passageway is disposed centrally between the four corner passageways.

10. The guide device of claim 9 wherein the guide device is a guide cube.
